(19) United States
(12) Patent Application Publication
(10) Pub. No.: US 2025/0000883 A1
(43) Pub. Date: Jan. 2, 2025

(54) TREATING NEUROINFLAMMATORY CONDITIONS

(71) Applicant: AN2 Therapeutics, Inc., Menlo Park, CA (US)

(72) Inventors: Eric Edward EASOM, Atherton, CA (US); Vincent S. HERNANDEZ, Brownsville, CA (US); Michael Richard Kevin ALLEY, Santa Clara, CA (US)

(73) Assignee: AN2 Therapeutics, Inc., Menlo Park, CA (US)

(21) Appl. No.: 18/704,383

(22) PCT Filed: Oct. 27, 2022
(86) PCT No.: PCT/US2022/047990

§ 371 Date: Apr. 24, 2024

Related U.S. Application Data
(60) Provisional application No. 63/273,766, filed on Oct. 29, 2021.

(51) Int. Cl. A61K 31/69; A61P 31/04

(52) U.S. Cl. CPC A61K 31/69; A61P 31/04

(57) ABSTRACT

This invention relates to, among other items, treating Alzheimer's disease by administering epetraborole, or a salt, hydrate or solvate thereof, to a patient in need of treatment thereof.

### PRIORITY CLAIM

This application claims benefit to U.S. Provisional Application Ser. No. 63/273,766, filed Oct. 29, 2021, which is incorporated herein by reference in its entirety.

### BACKGROUND OF THE INVENTION

The global rise of bacteria and other microorganisms resistant to antibiotics and antimicrobials in general, poses a major threat. Deployment of massive quantities of antimicrobial agents into the ecosphere during the past 60 years has introduced a powerful selective pressure for the emergence and spread of antimicrobial-resistant pathogens. Thus, there is a need to discover new broad spectrum antimicrobials, such as antibiotics, useful in combating microorganisms, especially those with multidrug-resistance.

Boron-containing molecules, such as 1-hydroxy-1,3-dihydro-benzo[c][1,2]oxaborole (also sometimes known as 1-hydroxy-benzo[c][1,2]oxaborole or oxaboroles or cyclic boronic esters), useful as antimicrobials have been described previously, such as in U.S. patent applications Ser. No. 12/142,692; Ser. No. 11/505,591 and Ser. No. 11/357,687. Generally speaking, a 1-hydroxy-1,3-dihydro-benzo[c][1,2]oxaborole has the following structure and substituent numbering system:

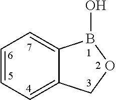

Surprisingly, it has now been discovered that certain classes of 1-hydroxy-1,3-dihydro-benzo[c][1,2]oxaboroles are effective antibacterials. This, and other uses of these 1-hydroxy-1,3-dihydro-benzo[c][1,2]oxaboroles are described herein.

### SUMMARY OF THE INVENTION

In a first aspect, the invention provides a method of treating a neuroinflammatory condition comprising administering epetraborole, or a salt, hydrate, or solvate thereof, to a patient in need of treatment thereof, thereby treating the neuroinflammatory condition. In an exemplary embodiment, the neuroinflammatory condition is Alzheimer's disease. In an exemplary embodiment, the invention provides a method of treating Alzheimer's disease comprising administering epetraborole, or a salt, hydrate, or solvate thereof, to a patient in need of treatment thereof, thereby treating Alzheimer's disease. In an exemplary embodiment, the patient is a human.

### DETAILED DESCRIPTION OF THE INVENTION

### I. Definitions and Abbreviations

As used herein, the singular forms “a,” “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxy carbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino) pyridine; DME is 1,2-dimethoxyethane; DMF is N,N-dimethylformamide; DMSO is dimethylsulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′,N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl)amide; KHMDS is potassium bis(trimethylsilyl)amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino) ferrocene]dichloropalladium (II); Pd/C is the catalyst known as palladium on carbon; Pd₂(dba)₃ is an organometallic catalyst known as tris(dibenzylideneacetone) dipalladium(0); Ra Ni or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂-PhSO₂Cl is 3-fluoro-4-nitrophenylsulfonyl chloride; 2-MeO-4-NO₂-PhSO₂Cl is 2-methoxy-4-nitrophenylsulfonyl chloride; and (EtO)₂POCH₂COOEt is a triethylester of phosphonoacetic acid known as triethyl phosphonoacetate.

“Epetraborole of the invention,” as used herein refers to epetraborole, salts (e.g. pharmaceutically acceptable salts), solvates and hydrates of these compounds.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono-or polyunsaturated and can include di-and multivalent radicals, having the number of carbon atoms designated (i.e. C₁-C₁₀ means one to ten carbons). In some embodiments, the term “alkyl” means a straight or branched chain, or combinations thereof, which may be fully saturated, mono-or polyunsaturated and can include di-and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1-and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—. Typically, an alkyl (or alkylene) group will have from 1 to 24 carbon atoms, with those groups having 10 or fewer carbon atoms being preferred in the invention. A “lower alkyl” or “lower alkylene” is a shorter chain alkyl or alkylene group, generally having eight or fewer carbon atoms.

The term “alkenylene” by itself or as part of another substituent means a divalent radical derived from an alkene.

The term “cycloalkylene” by itself or as part of another substituent means a divalent radical derived from a cycloalkyl.

The term “heteroalkylene” by itself or as part of another substituent means a divalent radical derived from an heteroalkane.

The term “heterocycloalkylene” by itself or as part of another substituent means a divalent radical derived from an heterocycloalkane.

The term “arylene” by itself or as part of another substituent means a divalent radical derived from an aryl.

The term “heteroarylene” by itself or as part of another substituent means a divalent radical derived from heteroaryl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 or 2 or 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes those radicals in which an aryl group is attached through the next moiety to the rest of the molecule. Thus, the term “arylalkyl” is meant to include those radicals in which an aryl group is attached to an alkyl group (e.g., benzyl, 1-(3-nitrophenyl) ethyl and the like). A substituent such as benzyl or 1-(3-nitrophenyl) ethyl can also be represented by ‘substituted alkyl’ wherein the ethyl radical is substituted with a 3-nitrophenyl moiety. The term “aryloxy” is meant to include those radicals in which an aryl group is attached to an oxygen atom. The term “aryloxyalkyl” is meant to include those radicals in which an aryl group is attached to an oxygen atom which is then attached to an alkyl group (e.g., phenoxymethyl, 3-(1-naphthyloxy) propyl, and the like).

For brevity, the term “heteroaryl” when used in combination with other terms (e.g., heteroaryloxy, heteroarylthioxy, heteroarylalkyl) includes those radicals in which a heteroaryl group is attached through the next moiety to the rest of the molecule. Thus, the term “heteroarylalkyl” is meant to include those radicals in which a heteroaryl group is attached to an alkyl group (e.g., pyridylmethyl and the like). The term “heteroaryloxy” is meant to include those radicals in which a heteroaryl group is attached to an oxygen atom. The term “heteroaryloxyalkyl” is meant to include those radicals in which an aryl group is attached to an oxygen atom which is then attached to an alkyl group. (e.g., 2-pyridyloxymethyl and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR″″′—C(NR′R″R′″)═NR″″, —NR″″—C(NR′R″)═NR″′, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2m′+1), where m′ is the total number of carbon atoms in such radical. R′, R″, R″′, R″″ and R″″′ each preferably independently refer to hydrogen, substituted or unsubstituted heteroalkyl, substituted or unsubstituted aryl, e.g., aryl substituted with 1 or 2 or 3 halogens, substituted or unsubstituted alkyl, alkoxy or thioalkoxy groups, or arylalkyl groups. When a compound of the invention includes more than one R group, for example, each of the R groups is independently selected as are each R′, R″, R″′, R″″ and R″″′ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR″″′—C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′, R″″ and R″″′ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R″′, R″″ and R″″′ groups when more than one of these groups is present.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer from 1 or 2 or 3 or 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers from 0 or 1 or 2 or 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁ or C₂ or C₃ or C₄ or C₅ or C₆) alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 or 6 or 7 atoms in the encircling arrangement. Unless otherwise specified, the ring optionally includes a heteroatom. Thus, the term “5- to 7-membered ring” includes, for example phenyl, pyridinyl and piperidinyl. The term “5- to 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur(S), silicon (Si), and boron (B).

The term “leaving group” means a functional group or atom which can be

displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired result.

The term “pharmaceutically acceptable salt” is meant to include a salt of an epetraborole of the invention which is prepared with relatively nontoxic acids or bases, depending on the particular substituents found on the compounds described herein. When epetraborole of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, l-arginine, d-lysine or l-lysine), or magnesium salt, or a similar salt. When epetraborole of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66:1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62:114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis-and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S′)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

The term “excipients” is conventionally known to mean carriers, diluents and/or vehicles used in formulating drug compositions effective for the desired use.

The term “microbial infection” or “infection by a microorganism” refers to any infection of a host tissue by an infectious agent including, but not limited to, bacteria or protozoa (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of enzyme. In an exemplary embodiment, the enzyme is an editing domain of a tRNA synthetase.

Boron is able to form additional covalent or dative bonds with oxygen, sulfur or nitrogen under some circumstances in this invention.

Embodiments of the invention also encompass compounds that are poly-or multi-valent species, including, for example, species such as dimers, trimers, tetramers and higher homologs of the compounds of use in the invention or reactive analogues thereof.

“Salt counterion”, as used herein, refers to positively charged ions that associate with a compound of the invention when the boron is fully negatively or partially negatively charged. Examples of salt counterions include H⁺, H₃O⁺, ammonium, potassium, calcium, magnesium (such as choline or diethylamine or amino acids such as d-arginine, l-arginine, d-lysine or l-lysine) and sodium.

The compounds comprising a boron bonded to a carbon and three heteroatoms (such as three oxygens described in this section) can optionally contain a fully negatively charged boron or partially negatively charged boron. Due to the negative charge, a positively charged counterion may associate with this compound, thus forming a salt. Examples of salt counterions include H⁺, H₃O⁺, ammonium, potassium, calcium, magnesium (such as choline or diethylamine or amino acids such as d-arginine, l-arginine, d-lysine or l-lysine) and sodium. The salts of the compounds are implicitly contained in descriptions of these compounds.

### II. Introduction

The role of Porphyromonas gingivalis in neuroinflammatory conditions such as Alzheimer's disease has been described in the art, such as in Panza et al, Brain 2019:142; 2905-2929. The invention provides methods of treating Alzheimer's disease through administration of epetraborole, or a salt, hydrate or solvate thereof.

### III. Compound

### Epetraborole, or a Salt, Hydrate, or Solvate Thereof

Epetraborole has a structure according to the following formula:

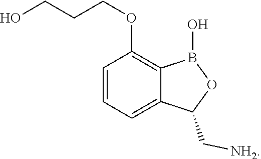

Epetraborole can be produced according the methods such as those disclosed in PCT Pat Pub WO 2008/157726 (PCT Pat App PCT/US2008/07550); U.S. Pat. No. 7,816,344 (U.S. patent application Ser. No. 12/142,692); PCT Pat Pub WO 2011/127143 (PCT Pat App PCT/US2011/031384); and U.S. Pat. No. 9,243,003 (U.S. patent application Ser. No. 13/639,594).

The epetraborole can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the invention provides epetraborole, or a salt, hydrate or solvate thereof, or a combination thereof. In an exemplary embodiment, the invention provides epetraborole, or a salt, hydrate or solvate thereof. In an exemplary embodiment, the invention provides epetraborole, or a salt thereof. In an exemplary embodiment, the salt is a pharmaceutically acceptable salt. In an exemplary embodiment, the invention provides a hydrochloride salt of epetraborole. In an exemplary embodiment, the invention provides epetraborole, or a hydrate thereof. In an exemplary embodiment, the invention provides epetraborole, or a solvate thereof.

### IV. Methods

In another aspect, the epetraborole of the invention exhibits potency against microorganisms, such as bacteria, and therefore has the potential to kill and/or inhibit the growth of microorganisms. In another aspect, the epetraborole of the invention exhibit potency against microorganisms, such as bacteria, and therefore have the potential to achieve therapeutic efficacy in the patients described herein.

In a further aspect, the invention provides a method of treating a microorganism infection, or a method of killing and/or inhibiting the growth of a microorganism, said method comprising: contacting said microorganism with an effective amount of an epetraborole of the invention, thereby killing and/or inhibiting the growth of the microorganism.

In a further aspect, the invention provides a method of treating a bacterial infection comprising administering to an animal suffering from the infection an effective amount of an epetraborole of the invention, or a pharmaceutically acceptable salt thereof, thereby treating the bacterial infection. In an exemplary embodiment, the invention provides a method of treating a bacterial infection comprising administering to an animal suffering from the infection an effective amount of an epetraborole of the invention, or a pharmaceutically acceptable salt thereof, and an effective amount of an antibiotic, or a pharmaceutically acceptable salt thereof, thereby treating the bacterial infection.

In an exemplary embodiment, the microorganism is a bacteria. In an exemplary embodiment, the epetraborole is described herein, or a salt, prodrug, hydrate or solvate thereof, or a combination thereof. In an exemplary embodiment, the invention provides epetraborole, or a salt, hydrate or solvate thereof. In an exemplary embodiment, the invention provides epetraborole, or a salt thereof. In another exemplary embodiment, the invention provides epetraborole, or a pharmaceutically acceptable salt thereof. In an exemplary embodiment, the epetraborole is part of a pharmaceutical formulation described herein. In another exemplary embodiment, the contacting occurs under conditions which permit entry of the epetraborole into the organism. Such conditions are known to one skilled in the art and are described herein.

In another aspect, the microorganism is inside, or on the surface of an animal. In another exemplary embodiment, the animal is described herein. In another exemplary embodiment, the animal is a human.

In an exemplary embodiment, the microorganism infection is treated, or the microorganism is killed or its growth is inhibited, through oral administration of the epetraborole of the invention. In an exemplary embodiment, the microorganism infection is treated, or the microorganism is killed or its growth is inhibited through intravenous administration of the epetraborole of the invention.

In an exemplary embodiment, the microorganism is a bacterium. In an exemplary embodiment, an infection is caused by and/or associated with a microorganism, particularly a bacterium. In an exemplary embodiment, the bacterium is Porphyromonas gingivalis.

The epetraborole of the invention exhibit potency against microorganisms, such as bacteria, and therefore have the potential to be used to treat a microorganism infection, such as a bacterial infection.

In a further aspect, the invention provides a method of treating a bacterial infection comprising administering to an animal suffering from the infection an effective amount of a compound of the invention, or a pharmaceutically acceptable salt thereof, thereby treating the bacterial infection. In an exemplary embodiment, the invention provides a method of treating a bacterial infection comprising administering to an animal suffering from the infection an effective amount of a compound of the invention, or a pharmaceutically acceptable salt thereof, and an effective amount of an antibiotic, or a pharmaceutically acceptable salt thereof, thereby treating the bacterial infection.

The compounds of the invention exhibit potency against microorganisms, such as bacteria, and therefore have the potential to achieve therapeutic efficacy in the animals described herein.

In another aspect, the invention provides a method of treating a neuroinflammatory conditions. In an exemplary embodiment, the neuroinflammatory condition is Alzheimer's disease.

In an exemplary embodiment, the disease is treated through oral administration of a compound of the invention. In an exemplary embodiment, the disease is treated through intravenous administration of a compound of the invention. In an exemplary embodiment, the disease is treated through subcutaneous administration of a compound of the invention.

### V. Pharmaceutical Formulation

In another aspect, the invention provides a pharmaceutical formulation comprising: a) epetraborole or a salt, hydrate, or solvate thereof; and b) a pharmaceutically acceptable excipient.

It is to be understood that the present invention covers all combinations of aspects and/or embodiments, as well as suitable, convenient and preferred groups described herein.

The invention is further illustrated by the Examples that follow. The Examples are not intended to define or limit the scope of the invention.

### EXAMPLES

### Example 1

### Antibacterial MIC Testing

Epetraborole was placed is a stock solution and stored at −70° C. Serial two-fold dilutions were prepared on the day of testing.

### Bacterial Strains

Porphyromonas gingivalis organisms were recovered from clinical samples, identified by standard methods or by partial sequencing of the 16S gene and stored in 20% skim milk at −70° C. All isolates were taken from the freezer and subcultured at least twice on supplemented Brucella agar to ensure good growth. Inocula were prepared by direct suspensions of cells from 48 h cultures into saline or Brucella broth.

For all tested organisms, supplemented Brucella agar deeps were obtained from Anaerobe Systems (Morgan Hill, CA). Defibrinated sheep blood (Hardy Diagnostics, Santa Maria, CA) was frozen and thawed to produce laked blood. On the day of testing, laked blood and the antimicrobial agents were added to the tubes of molten agar before pouring the agar dilution plates. The strains were applied to the plates using a Steers multipronged inoculator for a final concentration of approximately 10⁵ cfu/spot. After 44 h incubation at 36° C. in the anaerobic chamber incubator, the plates were examined for growth, and the MICs interpreted. The MIC was the lowest concentration that completely inhibited growth or resulted in a marked reduction of growth compared to the drug-free growth control.

### Results

| RMA# | Organism | Specimen source | Date Isolated | Date Tested | MIC (μg/ml) |
|---|---|---|---|---|---|
| 15676 | Po. gingivalis | Abd-perit.fld | Aug. 20, 2003 | Oct. 15, 2010 | 2 |
| 15913 | Po. gingivalis | Abd-perit.fld | Oct. 18, 2003 | Oct. 15, 2010 | 2 |
| 16269 | Po. gingivalis | Abd-perit.fld | Jan. 28, 2004 | Oct. 15, 2010 | 0.125 |
| 16389 | Po. gingivalis | Abd-perit.fld | Feb. 6, 2004 | Oct. 15, 2010 | 0.5 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a neuroinflammatory condition in a human in need thereof, the method comprising administering epetraborole, or a salt, hydrate or solvate thereof, to the human.

2. The method of claim 1, wherein the neuroinflammatory condition is Alzheimer's disease.

3. (canceled)

4. The method of claim 1, wherein the epetraborole, or a pharmaceutically acceptable salt thereof, is administered to the human orally.

5. The method of claim 1, wherein the epetraborole, or a pharmaceutically acceptable salt thereof, is administered to the human intravenously.

6. The method of claim 1, wherein the epetraborole, or a pharmaceutically acceptable salt thereof, is administered to the human subcutaneously.

7. The method of claim 1, wherein the epetraborole, or a pharmaceutically acceptable salt thereof, is administered to the human.

8. A method of treating an infection by Porphyromonas gingivalis in a human suffering from a neuroinflammatory condition, the method comprising administering epetraborole, or a salt, hydrate or solvate thereof to the human.

9. The method of claim 8, wherein the human suffers from Alzheimer's disease.

10. The method of claim 8, wherein the epetraborole, or a pharmaceutically acceptable salt thereof, is administered to the human orally.

11. The method of claim 8, wherein the epetraborole, or a pharmaceutically acceptable salt thereof, is administered to the human intravenously.

12. The method of claim 8, wherein the epetraborole, or a pharmaceutically acceptable salt thereof, is administered to the human subcutaneously.

13. The method of claim 8, wherein the epetraborole, or a pharmaceutically acceptable salt thereof, is administered to the human.

14. A method of treating a neuroinflammatory condition caused by Porphyromonas gingivalis in a human in need thereof, the method comprising administering epetraborole, or a salt, hydrate or solvate thereof to the human.

15. The method of claim 14, wherein the neuroinflammatory condition is Alzheimer's disease.

16. The method of claim 14, wherein the epetraborole, or a pharmaceutically acceptable salt thereof, is administered to the human orally.

17. The method of claim 14, wherein the epetraborole, or a pharmaceutically acceptable salt thereof, is administered to the human intravenously.

18. The method of claim 14, wherein the epetraborole, or a pharmaceutically acceptable salt thereof, is administered to the human subcutaneously.

19. The method of claim 14, wherein the epetraborole, or a pharmaceutically acceptable salt thereof, is administered to the human.
